SEQUENCE LISTING (4 sequences)

SEQ ID NO: 1 - DNA, 19 bp, synthetic construct
  source 1..19: mol_type = other DNA
ctggtgacccgtgctgctt

SEQ ID NO: 2 - DNA, 19 bp, synthetic construct
  source 1..19: mol_type = other DNA
tttgccgccttctgcctta

SEQ ID NO: 3 - DNA, 20 bp, synthetic construct
  source 1..20: mol_type = other DNA
gaccaccaaacatgacacca

SEQ ID NO: 4 - DNA, 20 bp, synthetic construct
  source 1..20: mol_type = other DNA
catcggcgagtacagactca

(19) United States
(12) Patent Application Publication
(10) Pub. No.: US 2024/0423949 A1
(43) Pub. Date: Dec. 26, 2024

(54) APPLICATION OF SALVIANOLIC ACID B IN PREPARING ANTI-ROTAVIRUS PREPARATION

(71) Applicant: GUANGDONG MEDICAL UNIVERSITY, DONGGUAN (CN)

(72) Inventors: LIJUN SONG, DONGGUAN (CN); WENCHANG ZHAO, DONGGUAN (CN)

(21) Appl. No.: 18/648,742

(22) Filed: Apr. 29, 2024

(30) Foreign Application Priority Data
Jun. 20, 2023 (CN) 202310734123.7

(51) Int. Cl. A61K 31/343; A61P 31/14

(52) U.S. Cl. CPC A61K 31/343; A61P 31/14

(57) ABSTRACT

An application of an salvianolic acid B in preparing an anti-rotavirus preparation is disclosed. The salvianolic acid B has no drug toxicity to cells at 16-256 μM and can effectively inhibit the biosynthesis of rotavirus at ranges from 64 to 256 μM. Salvianolic acid B exerts the effect of inhibiting rotavirus biosynthesis by inhibiting the structural protein VP6. Salvianolic acid B has no adsorption and direct inhibitory effects on rotavirus, providing a new pathway for resisting rotavirus.

### TECHNICAL FIELD

The present disclosure relates to the field of pharmaceutical technology, more specifically to the application of salvianolic acid B in preparing an anti-rotavirus preparation.

### SEQUENCE LISTING

The instant application contains a Sequence Listing which has been submitted electronically in ASCII format and is hereby incorporated by reference in its entirety. Said ASCII copy, created on Jul. 15 2024, is named U.S. Ser. No. 18/648,742-SEQ.xml and is 4,766 bytes in size.

### BACKGROUND ART

Rotavirus (RV) is a non-enveloped double stranded RNA virus belonging to the family of Reoviridae, formed by three layers of concentric protein layers, enveloping a genome formed by 11 segments of double stranded RNA (dsRNA), and encoding six structural proteins (VP1-4, VP6, VP7) and six non-structural proteins (NSP1-6). RV is the main pathogen causing severe gastroenteritis in infants and young children, and the infection of RV may result in serious diarrhea, dehydration, and even death. At present, there is no specific drug for enteritis caused by RV. Vaccination is a main method for preventing rotavirus, but the effect is very limited in developing countries. Therefore, it is of great significance to investigate new drugs for preventing and treating RV infections.

### SUMMARY

In view of the above, the present disclosure provides an application of salvianolic acid B in preparing an anti-rotavirus preparation.

The chemical structure formula of Salvianolic acid B (Sal B) is as follows:

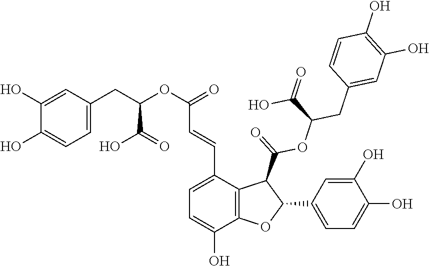 [SMILES: O=C(/C=C/c1ccc(O)c2c1[C@@H](C(=O)O[C@H](Cc1ccc(O)c(O)c1)C(=O)O)[C@H](c1ccc(O)c(O)c1)O2)O[C@H](Cc1ccc(O)c(O)c1)C(=O)O]

It is a water-soluble substance with high activity expression extracted from the roots and stems of the traditional Chinese medicine Danshen. The research of the present disclosure shows that salvianolic acid B achieves the effect of inhibiting rotavirus biosynthesis by inhibiting the expression of gene VP6 of rotavirus.

The application of salvianolic acid B in preparing a preparation against diseases caused by rotavirus is provided.

As a preferred technical solution, the effective concentration of salvianolic acid B is 64-256 μM.

The ability of salvianolic acid B to inhibit rotavirus biosynthesis is not significantly different from that of ribavirin, a positive drug, and when the effective concentration of salvianolic acid B is 64-256 μM, the inhibition rate is higher than 50%, and the effect of inhibiting rotavirus biosynthesis is significant.

More preferably, the effective concentration of salvianolic acid B is 256 μM.

Another object of the present disclosure is to provide an anti-rotavirus preparation, including salvianolic acid B.

As the preferred technical solution, the effective concentration of salvianolic acid B is 64-256 μM.

More preferably, the effective concentration of salvianolic acid B is 256 μM.

As a preferred technical solution, the preparation further includes pharmaceutically acceptable excipients.

As a preferred technical solution, the preparation is in a solid, semi-solid, or liquid form.

As a preferred technical solution, the preparation is a solution, a lozenge, a capsule, a tablet, a granule, a pill or a powder.

From the above technical solution, it can be seen that compared with the existing technology, the present disclosure provides the application of salvianolic acid B in preparing an anti-rotavirus preparation. Salvianolic acid B has no drug toxicity to cells in the concentration of 0 μM to 256 μM, and can effectively inhibit the biosynthesis of rotavirus in the concentration of 64 μM to 256 μM. Salvianolic acid B exerts an effect of inhibiting rotavirus biosynthesis of rotavirus by inhibiting structural protein VP6, without adsorption or direct inhibition on rotavirus, which provides a new route for anti-rotavirus preparation.

### DESCRIPTION OF THE ACCOMPANYING DRAWINGS

In order to explain the embodiments of the present disclosure or the technical solutions in the prior art more clearly, the following drawings that need to be used in the description of the embodiments or the prior art are briefly introduced. Obviously, the drawings in the following description are only embodiments of the present disclosure. For those of ordinary skill in the art, other drawings can be obtained based on the drawings disclosed without creative work.

FIG. 1 shows normal MA104 cells;

FIG. 2 shows MA104 cells infected with rotavirus for 48 hours;

FIG. 3 shows the toxicity results of salvianolic acid B on MA104 cells (compared to the untreated control group, ***P<0.001, ****P<0.0001);

FIG. 4 shows the inhibiting RV adsorption results of salvianolic acid B (compared with ribavirin group, ^{Δ}P<0.05);

FIG. 5 shows the inhibiting biosynthetic effect of salvianolic acid B on RV (no significant difference compared to the ribavirin group);

FIG. 6 shows the direct inhibitory effect of salvianolic acid B on RV (compared with Ribavirin group, ^{ΔΔΔ}<0.001);

FIG. 7 shows the effect of salvianolic acid B on the gene expression of viral structural protein VP6 in RV infected MA104 cells (compared with the RV group, ^{###}P<0.001, ^{####}P<0.0001);

Note: The experimental results of the present disclosure were plotted using GraphPad (Prism 8.0) software, and P<0.05 indicates that the difference is statistically significant. (***P<0.001, ****P<0.0001 indicates comparison with untreated control group; ^{Δ}P<0.05^{ΔΔΔ}P<0.001 indicates comparison with ribavirin group; ^{###}P<0.001, ^{####}P<0.0001 indicates comparison with RV group).

### DETAILED DESCRIPTION OF THE EMBODIMENTS

The following will provide a clear and complete description of the technical solution in the embodiments of the present disclosure, in conjunction with the accompanying drawings. Obviously, the described embodiments are only a part of the embodiments of the present disclosure, not all of them. Based on the embodiments in the present disclosure, all other embodiments obtained by ordinary technicians in the art without creative labor fall within the scope of protection of the present disclosure.

The embodiments of the present disclosure provide the application of salvianolic acid B in the preparing an anti-rotavirus preparation. All reagents used are commercially available and their sources are not specifically limited. The methods involved, unless otherwise mentioned, are conventional methods and will not be repeated here.

### Embodiment 1

### 1. Main Materials and Reagents for the Experiment

Salvianolic acid B standard: Shanghai Yuanye Biotechnology Co., Ltd., batch number: B20261.

Wa strain rotavirus, provided by the present laboratory (Pathogenic Biology Laboratory of Guangdong Medical University).

Rhesus monkey embryonic kidney cells (MA104 cells), derived from the cell bank of Sun Yat-Sen University.

Anti rotavirus VP6 antibody Abcam (Cambridge, UK).

### 2. Main Reagent Preparation Process

Salvianolic acid B stock solution: an appropriate amount of salvianolic acid B standard was weighed, dissolved in a cell-grade DMSO in a super-clean bench, added with an appropriate amount of high glucose DMEM culture medium for dilution, and mixed well until the final DMSO concentration did not exceed 0.5% as the standard. The drug solution was filter sterilized through a 0.22 μM filter using a disposable sterile syringe, and the salvianolic acid B stock solution was obtained.

Ribavirine:100 mg/mL of ribavirin and high glucose DMEM medium were diluted 1:100 to 1 mg/mL.

Complete culture medium (containing 10% fetal bovine serum, 1% double antibody): 50 mL FBS and 5 mL double antibody (anti penicillin streptomycin) were added to 450 mL high glucose DMEM culture medium in a super-clean bench, mixed well and then divided into 50 mL centrifuge tubes labelled, sealed with a sealing membrane and labelled for the date, and stored at 4° C.

20 μg/mL EDTA-free trypsin digestion solution: EDTA-free 0.25% trypsin digestion solution and high glucose DMEM culture medium were diluted to 20 μg/mL in a ratio of 1:125.

Virus growth maintenance solution: 20 μg/mL EDTA-free tryptic digestion solution and high glucose DMEM culture medium were diluted to 1 μg/mL in a ratio of 1:20.

MTT preparation: 250 mg of MTT powder was dissolved in 50 mL of PBS, sonicated until the MTT powder was dissolved, filtered with 0.22 m filter membrane, divided into 10 mL ep tubes, sealed with a sealing membrane and the date marked, and stored in a freezer at −20° C. protected from light.

### Embodiment 2

Morphological Changes in MA104 Cells Infected with Rotavirus

The RV virus was rapidly dissolved in a 37° C. water bath kettle, the dissolved virus and a 20 μg/mL EDTA-free trypsin digestion solution were mixed uniformly at a ratio of 1:1, placed in a CO₂ incubator for incubation for 30 min, the incubated virus solution was added to MA104 cells grown to 80%-90%, then 3 mL of virus growth maintenance solution was further added to the culture bottle, and placed in the CO₂ incubator for further culturing. Cytopathic effect (CPE) occurred on MA104 cells after virus infection. When the lesion degree reaches 75%, the virus was collected, frozen and thawed repeatedly in a refrigerator at −20° C. for 3 times. After low-temperature centrifugation, the supernatant was collected, and the supernatant was the amplified virus liquid. The above process was repeated to amplify the RV.

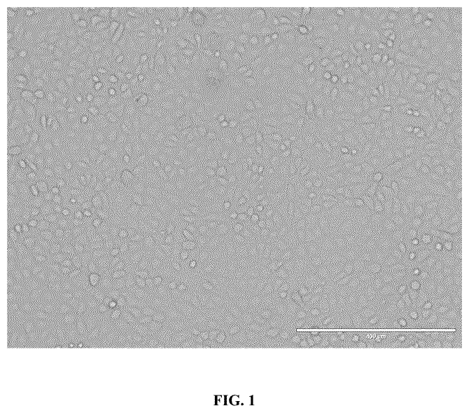

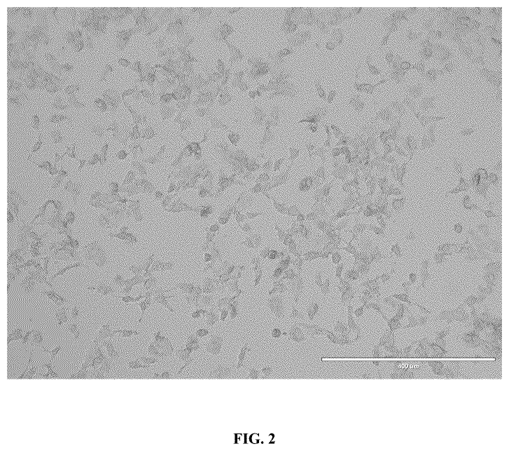

As shown in FIG. 1, the shape of normal MA104 cells was triangular or spindle shaped, with clear and distinct cell boundaries; as shown in FIG. 2, the MA104 cells infected with RV were significantly diseased, the cell boundaries became blurred, the distance between the cells increased, the black particles in the cells increased, and finally the cells completely fell off and floated.

### Embodiment 3

### Toxicity of Salvianolic Acid B on MA104 Cells (Detected by MTT Assay)

When the MA104 cells grew to monolayers, the cells were digested, centrifuged and resuspended, and the suspension was further diluted and then 10 μL was taken and counted on a blood cell counting plate, and the required cell volume was calculated. Cell plating was then performed in a 96-well plate with 100 μL of the cell suspension per well at a cell density of 8×10⁴ per mL. Cells grown to monolayers were treated with different concentrations of salvianolic acid B for 72 h, and the toxicity of salvianolic acid B on MA104 cells was detected by MTT assay. 10 μL of MTT solution was added to each well, and the mixture was incubated in the incubator for 3-4 h. Then, the 96-well plate was taken out, and the culture solution in the well was carefully aspirated from the wells. 150 μL of DMSO was added to each well, and the mixture was shaken at a low speed for 10 min on a shaker to fully dissolve the crystal, and the absorbance was detected and recorded at a wavelength of 490 nm.

The formula for the relative cell survival rate is:

Cell survival rate={(A_{experimental group}−A_{blank})/(A_{control group}−A_{blank})×100%}

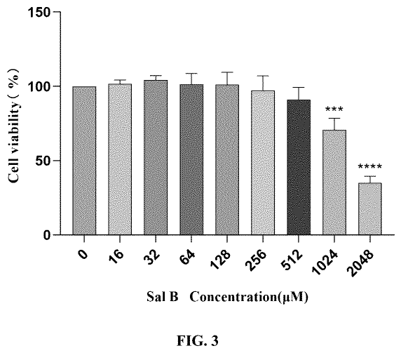

As shown in FIG. 3, when the concentration of salvianolic acid B was 512 μM, the cell survival rate was below 95% and toxicity began to occur. There was no obvious toxicity to MA104 cells in the concentration range of 0-256 μM, so the concentration within 16-256 μM can be selected as the dosing concentration for subsequent experiments.

### Embodiment 4

### The Mechanism of Salvianolic Acid B Against Rotavirus

In order to investigate whether salvianolic acid B has the effect of anti-RV infection in vitro, the adsorption, biosynthesis, and direct inhibition of salvianolic acid B against RV were studied in a MA104 cell model.

### 1. The Anti-RV Adsorption Effect of Salvianolic Acid B

Salvianolic acid B solutions of different concentrations were added into a 96-well culture plate of MA104 cells grown into monolayers, wherein 4 wells were repeated for each concentration of the solution, with 100 μL per well. An equal volume of ribavirin was added to the positive control group, and only an equal volume of DMEM culture solution was added to both the normal cell control group and the virus control group, and same were placed in a 5% CO₂ incubator at 37° C. for incubation for 2 h. The solution were aspirated, 100TCID50 virus solution (virus with 20 μg/mL trypsin at 37° C. for 30 min) was added to each of the remaining groups except the normal cell control group, with 100 μL per well, and after incubation in the 5% CO₂ incubator at 37° C. for incubation for 2 h, the virus was aspirated, the maintenance solution was added, with 100 μL per well, and after the 96-well was plated in the 5% CO₂ incubator at 37° C. for incubation and continuous observation for 72 hours, the MTT assay was used for detection. 1/10 volume of MTT solution was added to each well, after being incubated for 3-4 h in the incubator, the 96-well plate was taken out, the culture solution in the well was carefully aspirated, 150 uL of DMSO was added to each well, and same was placed on a shaker and shaken at a low speed for 10 min, so that the crystal was fully dissolved; and the absorbance was detected and recorded at a wavelength of 490 nm. The drug-to-virus inhibition rates were calculated and the experiment was repeated 3 times.

Virus inhibition rate (%)=(average A value of drug group−average A value of virus control group)/(average A value of normal cell control group−average A value of virus control group)×100%

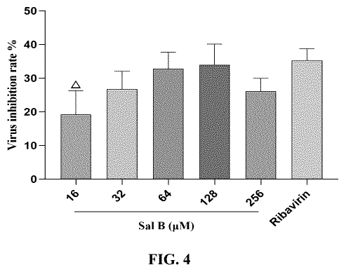

As shown in FIG. 4, compared with the ribavirin group, salvianolic acid B showed statistical differences at 16 μM, and there was no significant difference in its anti-RV adsorption effect compared to ribavirin in the concentration range of 32-256 μM, but the inhibition rates were all below 50%, indicating that salvianolic acid B had no significant anti-RV adsorption effect.

### 2. The Anti-RV Biosynthesis Effect of Salvianolic Acid B

100 TCID50 virus solution (virus with 20 μg/mL trypsin at 37° C. for 30 min) was added to a 96-well culture plate of MA104 cells grown to monolayers, with 100 μL per well, and the cells were rinsed twice with PBS before addition. A normal cell control group was set, an equal volume of DMEM culture solution was added, then the 96-well plate was placed in a 5% CO₂ incubator at 37° C. for incubation for 2 h, the virus solution was aspirated, different concentrations of salvianolic acid B and ribavirin were added, respectively, with 100 μL per well, and a virus control group was set and added with only a maintenance solution, with 100 μL per well. The 96-well plate was placed in a 5% CO₂ incubator at 37° C. for incubation and continuous observation for 72 h and then detected by the MTT assay. A 1/10 volume of MTT solution was added to each well. After being incubated for 3-4 h in the incubator, the 96-well plate was removed, the culture solution in the well was carefully aspirated, and 150 μL of DMSO was added to each well. The plate was shaken at a low speed for 10 min on a shaker to fully dissolve the crystal, and the absorbance was detected and recorded at a wavelength of 490 nm. The drug-to-virus inhibition rates were calculated and the experiment was repeated 3 times.

Virus inhibition rate (%)=(average A value of drug group−average A value of virus control group)/(average A value of normal cell control group−average A value of virus control group)×100%

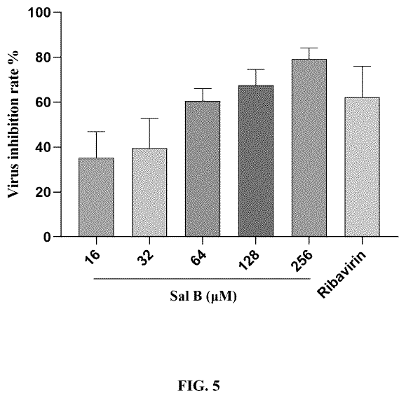

As shown in FIG. 5, compared with the ribavirin group, there was no significant difference in the drug groups with different concentrations of salvianolic acid B. However, the inhibitory effect on RV biosynthesis increased with the increase of drug concentration, showing a clear dose-response relationship. Among them, the inhibition rate of salvianolic acid B was higher than 50% at a concentration of 64-256 μM, and it had a significant inhibiting biosynthesis effect on RV.

### 3. Direct Inhibitory Effect of Salvianolic Acid B on RV

Different concentrations of salvianolic acid B solution with 50TCID50 virus solution (virus with 20 μg/mL trypsin at 37° C. for 30 min), DMEM with 50TCID50 virus solution, ribavirin with 50TCID50 virus solution were mixed in equal volumes and incubated in a 5% CO₂ incubator at 37° C. for 2 hours. The cells were rinsed twice with PBS and added to a 96-well culture plate of MA104 cells grown to monolayers, and for the normal cell control group, an equal volume of DMEM was added. After incubated at 37° C. and 5% CO2 for 2 h, the mixed solution was aspirated and the maintenance solution was added, with 100 μL per well. The 96-well plate was placed in a 5% CO₂ incubator at 37° C. for continuous observation for 72 h and then detected by the MTT assay. A 1/10 volume of MTT solution was added to each well. After incubated for 3-4 h in the incubator, the 96-well plate was removed, the culture solution in the well was carefully aspirated, and 150 L of DMSO was added to each well. The plate was shaken at a low speed for 10 min on a shaker to fully dissolve the crystal, and the absorbance was detected and recorded at a wavelength of 490 nm. The drug-to-virus inhibition rates were calculated and the experiment was repeated 3 times.

Virus inhibition rate (%)=(average A value of drug group−average A value of virus control group)/(average A value of normal cell control group−average A value of virus control group)×100%

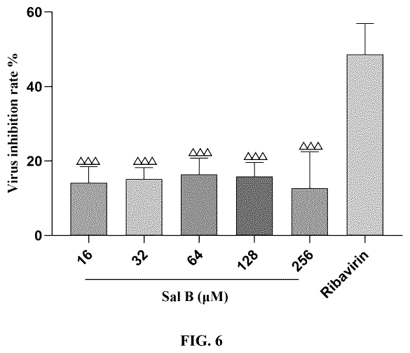

As shown in FIG. 6, compared with the ribavirin group, the inhibitory rates of salvianolic acid B directly inhibiting RV in the concentration range of 16-256 μM were 14.18%, 15.18%, 16.47%, 15.82%, and 12.71%, respectively, with statistical differences. And the inhibition rates of salvianolic acid B on RV were lower, all below 50%, indicating that salvianolic acid B had no direct inhibitory effect on RV.

### Embodiment 5

### The Effect of Salvianolic Acid B on the Gene Expression Level of RV Structural Protein VP6

### (1) Extraction and Quantification of Total RNA

\textcircled{1} In order to further verify whether salvianolic acid B has an inhibitory effect on RV synthesis, a drug group (MA104 cells after RV infection were treated with different concentrations of salvianolic acid B), a ribavirin group, a normal group, and an RV group were set up. The supernatant was removed and PBS rinse was performed twice. 1 mL of Trizol reagent was added to each well and allowed to stand for 5 minutes. After blowing the cells with a gun tip, they were collected in a 1.5 mL enzyme free EP tube. 200 μL of chloroform was add to the tube. After 15 seconds of vortex shock, the tube was placed at room temperature for 5 min, and centrifuged (4° C., 12000 r/min for 15 min). The centrifuged sample was divided into three layers, namely, the colorless upper layer, the white middle layer and the red lower layer.

\textcircled{2} The supernatant was carefully pipetted into a new 1.5 mL enzyme free EP tube (with a volume of approximately 400 μL), an equal volume of pre-cooled isopropanol was added, the mixture was vigorously shaken and mixed up and down, and centrifugation was performed (4° C., 12000 r/min, 15 min).

\textcircled{3} After centrifugation, white precipitate can be seen at the bottom of the EP tube. The supernatant was removed and the precipitate was retained, 1 mL of prepared 75% ethanol solution (prepared according to a 3:1 ratio using anhydrous ethanol and anhydrous enzyme) was added, the mixture was shaken and blended, centrifuged (4° C., 12,000 r/min, 10 min), and after the supernatant was discarded, the mixture is left at room temperature for 15 min-20 min and left to dry.

\textcircled{4} After drying, 20 μL of DEPC-treated water was added to the EP tube and the tube wall was gently blown to dissolve RNA. A NanoDrop microultraviolet spectrophotometer may be directly used in an experiment or stored at −80° C. for use after measuring the RNA concentration of the sample.

(2) Reverse Transcription Reaction of mRNA

\textcircled{1} Removal of Genomic DNA Reaction

A reaction mixture was prepared on ice according to the following ingredients with a reaction volume of 20 μL and the procedure was performed according to the instruction Evo M-MLV RT Kit with gDNA Clean for qPCR II. The consumables used in this experiment were Axygen enzyme free.

TABLE 1
Degenomic DNA reaction system

| Component Name | Addition |
|---|---|
| gDNA Clean Reagent | 1 μL |
| 5 × gDNA Clean Buffer | 2 μL |
| Total RNA*¹ | — |
| RNase free water | Up to 10 μL |

*1: The amount of RNA can be added as needed. In a 20 μL reverse transcription system, a maximum of 1 μg total RNA was used; When using the probe method, a maximum of 2 μg total RNA should be used.

Reaction conditions: 42° C. for 2 min, 4° C.

### \textcircled{2} Reverse Transcription Reaction

The reaction solution was prepared according to the table below and the reverse transcription reaction was performed.

TABLE 2
Reverse transcription reaction system

| Component Name | Addition |
|---|---|
| Reaction solution in Step 1) | 10 μL |
| Evo M-MLV RTase Enzyme Mix | 1 μL |
| Oligo dT(18T) Primer(50 μM) ⁺¹ | 1 μL |
| Random 6 mers Primer(400 μM) *¹ | 1 μL |
| 5 × RTase Reaction Buffer Mix I | 4 μL |
| RNase free water | 3 μL |
| Total | 20 μL |

Reaction conditions: 37° C. for 15 min, 85° C. for 5 sec, 4° C.

### (3) Real Time PCR Reaction

Real time quantitative PCR was performed using SYBR Green I fluorescence labeling to detect the expression levels of VP6 in the drug group, ribavirin group, normal group, and RV group, SYBR® Premix Pro Taq HS qPCR Kit II reagent kit was adopted, and GAPDH was selected as an internal reference. In the experiment, a real-time fluorescent quantitative PCR amplification reaction system was prepared according to the following table with a dedicated Real-time PCR plate of Axygen, and the reaction solution was prepared on ice (the total reaction was is 10 μL).

TABLE 3
qPCR reaction system

| Component Name | 10 μL system | |
|---|---|---|
| 2 × SYBR ® Green Pro Taq HS Premix II | 5 | μL |
| cDNA | 1 | μL |
| Primer F | 0.2 | μL |
| Primer R | 0.2 | μL |

| | |
|---|---|
| RNase free water | Up to 10 μL |

TABLE 4
qPCR reaction conditions

| | Temperature | Time | Number of cycles |
|---|---|---|---|
| Step 1 | 95° C. | 30 sec | 1 |
| Step 2 | 95° C. | 5 sec | 40 |
| | 60° C. | 30 sec | |
| Step 3 | Dissociation stage | | |

TABLE 5
Primer sequences

| Gene | | Primer (5′-3′) |
|---|---|---|
| GAPDH | Forward | CTGGTGACCCGTGCTGCTT (SEQ ID NO. 1) |
| | Reverse | TTTGCCGCCTTCTGCCTTA (SEQ ID NO. 2) |
| VP6 | Forward | GACCACCAAACATGACACCA (SEQ ID NO. 3) |
| | Reverse | CATCGGCGAGTACAGACTCA (SEQ ID NO. 4) |

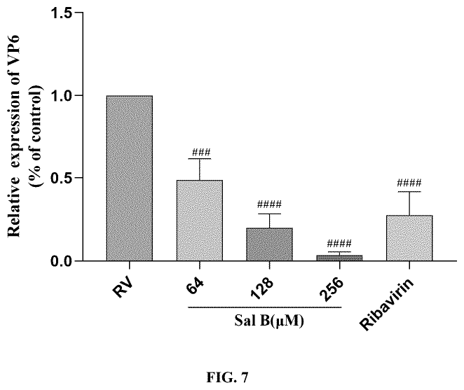

As shown in FIG. 7, the gene expression of VP6 in the ribavirin group was significantly reduced compared to the RV group, with a statistical difference (####P<0.001), indicating that ribavirin has an anti RV effect. Compared with the RV group, the expression level of VP6 in the salvianolic acid B group was significantly reduced at concentrations of 64, 128, and 256 μM, with statistical differences and a dose-response correlation. Among them, salvianolic acid B inhibited the expression of VP6 most significantly at 256 μM (###P<0.001), indicating that salvianolic acid B can exert an anti RV effect by inhibiting the gene expression of VP6.

The embodiments in this description are described in a progressive manner, each embodiment focuses on a difference from other embodiments, and reference may be made to each other for the same or similar parts of the embodiments.

The foregoing descriptions of the disclosed embodiments enable persons skilled in the art to implement or use the present disclosure. Various modifications to these embodiments will be readily apparent to those skilled in the art. The general principles defined herein may be implemented in other embodiments without departing from the spirit or scope of the disclosure. Therefore, the present disclosure will not be limited to the embodiments described herein but is to be accorded the widest scope consistent with the principles and novel features disclosed herein.

What is claimed is:

1. A method for preparing an anti-rotavirus preparation, comprising:
mixing a salvianolic acid B of an effective concentration of 64-256 μM with pharmaceutically acceptable excipients.

2. A method for curing gastroenteritis in infants and young children, comprising:
administrating an anti-rotavirus preparation into infants and young children; wherein the anti-rotavirus preparation comprises a salvianolic acid B of an effective concentration of 64-256 μM and pharmaceutically acceptable excipients.

3. The method of claim 1, wherein an effective concentration of the salvianolic acid B is 256 μM.

4. An anti-rotavirus preparation, wherein the anti-rotavirus preparation comprises a salvianolic acid B.

5. The anti-rotavirus preparation of claim 4, wherein an effective concentration of the salvianolic acid B is 64-256 μM.

6. The anti-rotavirus preparation of claim 4, wherein an effective concentration of the salvianolic acid B is 256 μM.

7. The anti-rotavirus preparation of claim 4, wherein the anti-rotavirus preparation further comprises pharmaceutically acceptable excipients.

8. The anti-rotavirus preparation of claim 4, wherein the anti-rotavirus preparation is in a solid, semi-solid, or liquid form.

9. The anti-rotavirus preparation of claim 4, wherein the anti-rotavirus preparation is a solution, a lozenge, a capsule, a tablet, a granule, a pill or a powder.
